(19) United States
(12) Patent Application Publication
(10) Pub. No.: US 2005/0038403 A1
(43) Pub. Date: Feb. 17, 2005

(54) Disposable potty garment

(76) Inventor: Deidre M. Singleton, Killeen, TX (US)

Correspondence Address: LITMAN LAW OFFICES, LTD., P.O. BOX 15035 CRYSTAL CITY STATION, ARLINGTON, VA, 22215, US

(21) Appl. No.: 10/638,638

(22) Filed: Aug. 12, 2003

(51) Int. Cl. A61F 13/15; A61F 13/20

(52) U.S. Cl. 604/385.13; 604/358

(57) ABSTRACT

A disposable potty garment, worn by a user with an upper leg area, a crotch area, a buttocks area, and two side hip areas. The invention has a generally flat and rectangular diaper that has a front end, a back end, a left side edge with a center area and a right side edge with a center area. The diaper is fitted with elastic gathers along the left side edge of the center area and the right side edge of the center area to accommodate and comfortably fit around the user's upper leg area. The diaper can also be balled up and disposed of in a plastic bag that is provided with the disposable potty garment.

### BACKGROUND OF THE INVENTION

1. Field of the Invention

The present invention relates to a disposable potty garment. More particularly, the invention is a disposable garment that can be worn and soiled into, and then discretely folded up and discarded.

2. Description of the Related Art

Incontinence garments are invaluable to those that suffer from incontinence. Incontinence garments can prevent embarrassing accidents during the normal course of the day for users and for persons who wet their beds at night. These garments are well represented in the related art.

United States Patent Application Publication Number 2001/0004689 published on Jun. 21, 2001, outlines a disposable diaper that includes a topsheet, a backsheet and a core disposed therebetween to define front and rear waist regions and a crotch region, and an elastic member extending to surround a predetermined zone of the core about the vicinity of a longitudinal center line that is placed in a rear half of the diaper defined between the vicinity of a transverse center line and the end of the rear waist region and secured under tension to the upper surface of the core so that the zone of the core is depressed downward to form a depression as the elastic member contracts.

United States Patent Application Publication Number 2002/0029029 published on Mar. 7, 2002, outlines the use of a disposable wearing article that includes a liquid-absorbent first core disposed between topsheets and backsheets, a pair of side flaps extending in a longitudinal direction outside transversely opposite side edges of the first core in a crotch region and a second core spaced from the respective side edges of the first core and disposed between the topsheets and the backsheets.

United States Patent Application Publication Number 2002/0072728 published on Jun. 13, 2002, outlines the use of a disposable undergarment with a topsheet, a backsheet and an absorbent core disposed therebetween. The core is placed in a crotch region having front and rear end zones and a middle zone. Bath side portions of a first elastic member extend in the vicinity of the front ends of transversely opposite side edge portions while a middle portion of the first elastic member extends across the front end zone of the core.

United States Patent Application Publication Number 2002/0087140 published on Jul. 4, 2002, outlines the use of a disposable body fluid absorbent wearing article which includes a body fluid absorbent pad, and a crotch region of the pad which is defined by a pair of darts extending in a back-and-forth direction of the article. The article has a body fluid absorbent core divided into an inner portion extending between the darts and the outer portions extending outside the darts.

United States Patent Application Publication Number 2002/0138060 published on Sep. 26, 2002, outlines the use of a disposable diaper with elastic threads for raisable gathers and elastic threads for leg gathers that are provided in a specified relationship in side sheets provided at the opposite sides of a skin-side sheet and the side sheets, which are adhered to the skin-side sheet in a specified relationship. In this way, the raisable gathers and leg gathers can be simultaneously formed at side sheets.

United States Patent Application Publication Number 2002/0138061 published on Sep. 26, 2002, outlines the use of a disposable diaper in which an absorbent body is provided between a water-repellent outer sheet and a hydrophilic skin-side sheet made of a nonwoven fabric. Raisable strips made of nonwoven fabric are provided at the opposite sides of the skin-side sheet, elastic threads for raisable gathers are provided in inner edge portions of the raisable strips and the outer edge portions of the raisable strips are adhered to the skin-side sheet. An ooze preventing portion for preventing an ooze of urine and other bodily waste is formed at opposite end portions of the skin-side sheet.

United States Patent Application Publication Number 2002/0151860 published on Oct. 17, 2002, outlines the use of a disposable absorbent article partially defined by a pair of longitudinally extending side edges and a pair of end edges extending between the side edges, which include a topsheet layer, a backsheet layer and an absorbent core disposed between the topsheet and the backsheet. The disposable absorbent article also has a pair of side wall structures with each having a pair of oppositely positioned end portions.

United States Patent Application Publication Number 2002/0173763 published on Nov. 21, 2002, outlines the use of a disposable liquid-absorbent pad that includes a liquid-pervious topsheet, a liquid-pervious backsheet and a liquid-absorbent core interposed between the two sheets. The pad is folded inwardly in the vicinity of transversely opposite side edges with the topsheet inside in a rear region. The pad is also folded outwardly in a transverse direction along folding lines extending in a longitudinal direction and is joined together along the folding lines so that the rear region of the pad defines an excretion receiving space extending upward and rearward.

United States Patent Application Publication Number 2002/0173764 published on Nov. 21, 2002, outlines the use of a pants-type disposable wearing article, which has a holder and an absorbent pad. The absorbent pad has a crotch region extending to the front and rear waist regions of the holder and has longitudinal front and rear ends attached to the front and rear waste regions. The absorbent pad has a liquid absorbent core and is provided with leak-barrier flaps rising from the side edges of the core and ribbon-like suspenders attached to the top edges of the leak-barrier flaps.

United States Patent Application Publication Number 2002/0173765 published on Nov. 21, 2002, outlines the use of an absorbent garment and a method for making an absorbent garment. The absorbent garment is provided with elastic side belts attached at an angle to the main body of the garment to provide a better fit to the user. Adjustment strips are affixed to the elastic side belts and to the main body of the garment to allow the user to repeatedly adjust the garment being worn.

United States Patent Application Publication Number 2002/0183713 published on Dec. 5, 2002, outlines the use of a disposable absorbent article having a pair of barrier leg cuffs and a pair of side panels. The barrier leg cuffs are disposed along opposing sides of an absorbent core and the side panels are disposed outboard of the barrier leg cuffs along opposing sides of the absorbent article. The barrier leg cuffs are integrally formed with the side panels from a single nonwoven ply.

United States Patent Application Publication Number 2002/0184698 published on Dec. 12, 2002, outlines the use of throw-away underwear designed to be a one-piece undergarment designed with a breathable, biodegradable material used in a manner similar to regular underwear. It is to be worn and then disposed of and is available for everyone regardless of age or gender.

United States Patent Application Publication Number 2003/0023220 published on Jan. 30, 2003, outlines the use of a pants-type disposable wearing article, which includes a chassis member and a liquid-absorbent member. A plurality of waist-surrounding elastic members extending in a waist-surrounding direction across the front and rear waist regions are spaced from one another at a given distance in a longitudinal direction and are secured in a stretched state to the chassis member, so that a plurality of gathers are formed on the chassis member as the elastic members contract.

United States Patent Application Publication Number 2003/0023225 published on Jan. 30, 2003, outlines the use of a disposable wearing article which includes a liquid-pervious topsheet, a liquid-pervious backsheet, a liquid-absorbent base panel interposed between these two sheets, a pair of side flaps lying outside transversely opposite side edges of the base panel so as to extend in a longitudinal direction and a pair of leak-barrier cuffs attached to the side flaps and elastic members secured to the distal side edge portions of the cuffs.

U.S. Pat. No. 4,302,853 issued to Mesek on Dec. 1, 1981, outlines the use of a disposable undergarment particularly suitable for the toilet training of infants. The undergarment is fabricated from a compressively shrunk non-woven fabric in a single layer or laminated layer to provide multiple layers. The undergarment is made from a single blank of substantially rectangular shape of which one longer side margin is adapted to become the waist-encircling portion of the undergarment, with the other side margin being adapted to become the leg openings.

U.S. Pat. No. 4,610,681 issued to Strohbeen et al. on Sep. 9, 1986, outlines the use of a disposable undergarment having a multi-layer web that is sealed with small, neat, exterior seams. The exterior seams are sealed along a plurality of narrow lines such that the total seam is less than about 3/16″ wide with the bonding area forming about 50′ of the total seal area.

U.S. Pat. No. 4,674,135 issued to Greene on Jun. 23, 1987, outlines the use of a disposable undergarment formed from a single sheet of non-woven cellulose material such as paper. The material is cut into a generally hour-glass shape defining a front portion, a back portion, an intermediate crotch portion, two generally semicircular opposed cut-outs at each side of the material, a detachable crotch insert portion formed within one of the opposed cut-outs, and may include a detachable circular container portion formed within one of the opposed cut-outs that are adjoined to the front portion.

U.S. Pat. No. 5,103,501 issued to Meiseis on Apr. 14, 1992, outlines the use of traveling and disposable underwear provided for three undergarments, which are an undershirt, a pair of men's underpants and a pair of women's panties. The three undergarments are made of an absorbent material and include elastic bands for comfortably holding the undergarments to the wearer. The three undergarments are also provided with magnetic fasteners for closing openings and securing together similarly provisioned garments, which allow for easier donning and doffing without tearing the undergarments.

U.S. Pat. No. 6,098,203 issued to Rajala et al. on Aug. 8, 2000, outlines the use of a three dimensional disposable garment, subassemblies of the garment, and an apparatus and methods for making the garment and subassemblies. The garment is designed to hold a primary absorbent sanitary pad. The garment may have elasticized leg and waist openings and be stretchable about the hip and stomach regions of a user.

U.S. Pat. No. 6,367,089 issued to Van Gompel et al. on Apr. 9, 2002, outlines the use of a three dimensional disposable panty for holding a sanitary pad. The panty may have elasticized leg and waist openings and be stretchable about the hip and stomach regions of a user. The panty provides backup leakage protection to the sanitary pad. The panty includes an absorbent barrier composite positioned in the crotch area that extends into the body of the disposable panty front and back and over the leg elastics to trap pad leakage inside the pant and prevent liquid strike through onto outer clothing and bed linen.

U.S. Pat. No. 6,423,048 issued to Suzuki et al. on Jul. 23, 2002, outlines the use of a disposable diaper which includes barrier side flaps extending along opposite side edges of a diaper body. The barrier side flaps each include a first side section extending above an upper side of the body and a second side section extending below a lower side of the body. The second side section includes being folded back with its inner surface put flat together so as to define a free subsection extending inward of the body and a fixed subsection extending outward of the body.

U.S. Pat. No. 6,497,693 issued to Otsubo on Dec. 24, 2002, outlines the use of a disposable undergarment such as a disposable diaper, which includes a front waist region, a rear waist region and a crotch region extending therebetween. The undergarment has bonded edges extending along a longitudinal center line of the rear waist region and a concavity depressed toward a surface destined to be remote from the wearer's skin. The rear waist region is divided into a first rear waist region and a second rear waste region along a longitudinal center line extending in the longitudinal direction to bisect a transverse dimension of the undergarment.

W.I.P.O. Pat. No. WO95/00096 granted to Bridges on Jan. 5, 1995, outlines the use of disposable training pants with non-perforated tear lines for removal and evacuation of contents. The non-perforated tear lines are a series of bond sites, which are shaped, sized, spaced and arranged to provide a zone of weakness to permit severance of nonelasticized portions as well as elastic disposal in the waistband, the body of the pant and the elastic leg bands for complete removal.

Web site www.etrode.com/category.cfm/category/136 is a Web site for Etrode, that features all through the night disposable briefs.

Web site www.hospeco.com/health/undqrmnts.asp is a Web site that features “At Ease Undergarments”.

Web site www.healthpursuit.com/undergarments.html is a Web site that features one size fits all disposable undergarments.

Although each of these patents and Web sites outline novel and useful incontinence garments, none of these devices can accommodate a user who must relieve himself or herself into an incontinence garment and discretely fold-up and discard it in a waste collection bag. Such an incontinence garment would be very useful for people who cannot relieve themselves because of their job that cannot be unattended to or due to a non-ambulatory health condition.

None of the above inventions and patents, taken either singularly or in combination, is seen to describe the instant invention as claimed. Thus a disposable potty garment solving the aforementioned problems is desired.

### SUMMARY OF THE INVENTION

The invention is a disposable potty garment, worn by a user with an upper leg area, a crotch area, a buttocks area, and two side hip areas. The invention has a generally flat and rectangular diaper that has a front end, a back end, a left side edge with a center area and a right side edge with a center area. The diaper is fitted with elastic gathers along the left side edge of the center area and the right side edge of the center area to accommodate and comfortably fit around the user's upper leg area. The diaper can be balled up and disposed of in a plastic bag that is provided with the disposable potty garment.

Accordingly, it is a principal object of the invention to provide a disposable potty garment that can be used while wearing, and then be discretely folded up and discarded.

It is another object of the invention to provide a disposable potty garment that could be used for patients who would normally use a bedpan.

It is a further object of the invention to provide a disposable potty garment that can be used by persons who do not have access to a bathroom.

It is an object of the invention to provide improved elements and arrangements thereof for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental, perspective view of a disposable potty garment according to the present invention.

FIG. 2 is an overhead perspective view of a disposable potty garment.

FIG. 3 is a front perspective view of a disposable potty garment being rolled up and being put in a plastic bag.

FIG. 4 is an overhead perspective view of an optional second layer used together with the disposable potty garment.

Similar reference characters denote corresponding features consistently throughout the attached drawings.

### DETAILED DESCRIPTION OF THE PREFERRED EMBODIMENTS

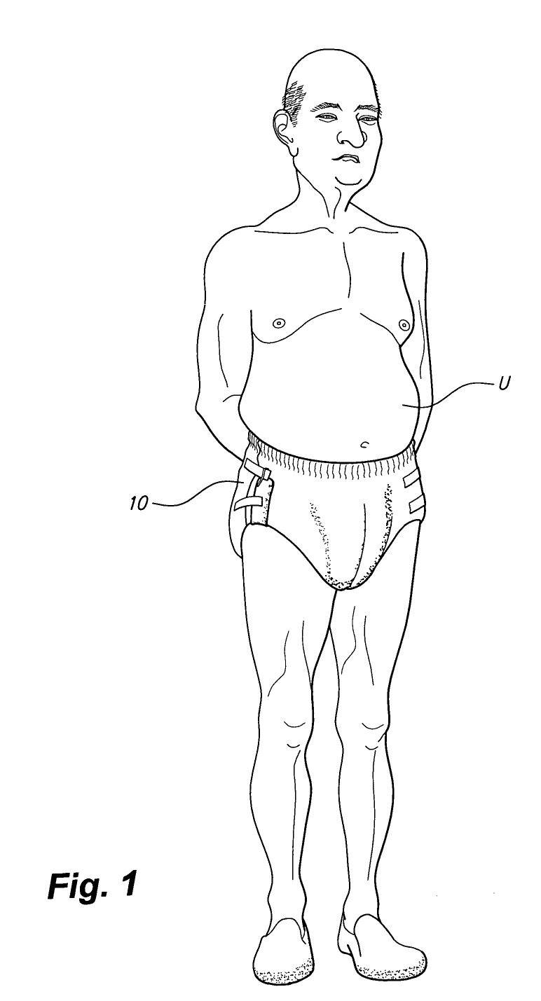

The present invention is a disposable potty garment 10, worn by a user U with an upper leg area, a crotch area, a buttocks area, and two side hip areas, to defecate and urinate into, as is shown in FIG. 1.

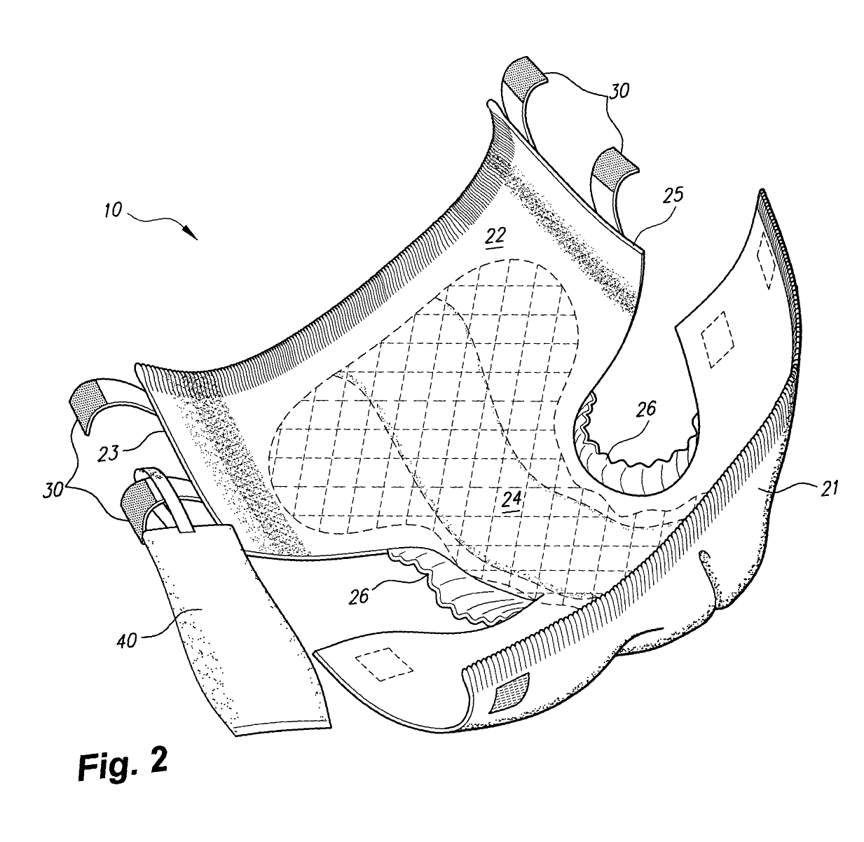

The disposable potty garment 10 comprises a generally flat and rectangular diaper 20 that has a front end 21, a back end 22, a left side edge 23 with a center area 24 and a right side edge 25 with a center area 24. The generally flat and rectangular diaper 20 is fitted with elastic gathers 26 along the left side edge 23 of the center area 24 and the right side edge 25 of the center area 24 to accommodate and comfortably fit around the user's U upper leg area. This is depicted in FIG. 2. The front end 21 of the generally flat and rectangular diaper 20 is pulled up to the user's U groin area while the rear end 22 of the generally flat and rectangular diaper 20 is wrapped back around underneath the user's U buttocks area. The front end 21 of the generally flat and rectangular diaper 20 is held together with the rear end 22 of the generally flat and rectangular diaper 20 on the side hip areas of the user U with a first plurality of hook and loop fasteners 30 securing the disposable potty garment 10 to the user U.

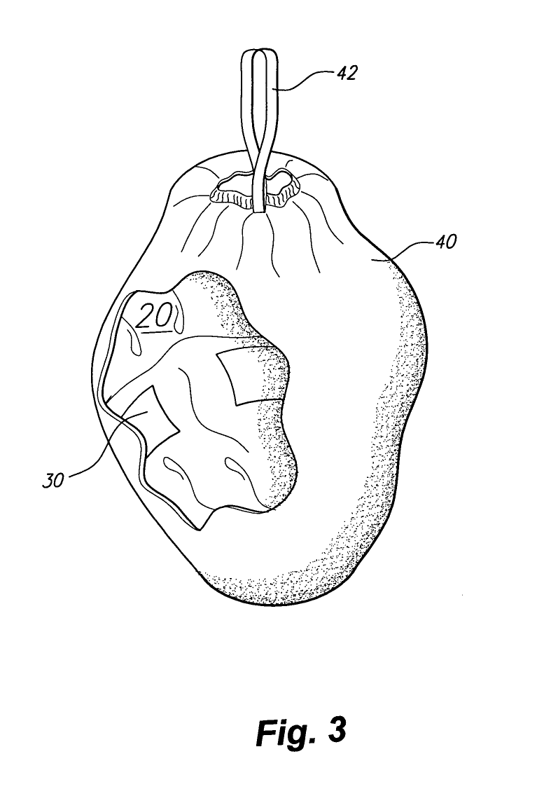

There are many incontinence garments in the related art that are designed to protect the user U wearing the garment from embarrassing sudden wetness or wetness occurring while the user U is asleep. The disposable potty garment 10 is designed to allow a user U to defecate or urinate into while wearing the disposable potty garment 10. The generally flat and rectangular diaper 20 can be folded up into a ball and held tightly together with the first plurality of hook and loop fasteners 30. The generally flat and rectangular diaper 20 can hold any human waste (not shown) defecated and urinated into by the user U. This is illustrated in FIG. 3. The folded up generally flat and rectangular diaper 20 and defecated and urinated human waste is disposed of in a plastic bag 40 that is provided with the disposable potty garment 10. The plastic bag 40 is conveniently provided with a close tying line 42 to allow a user U to tie off the top of the plastic bag 40 once it is filled with a soiled disposable potty garment 10.

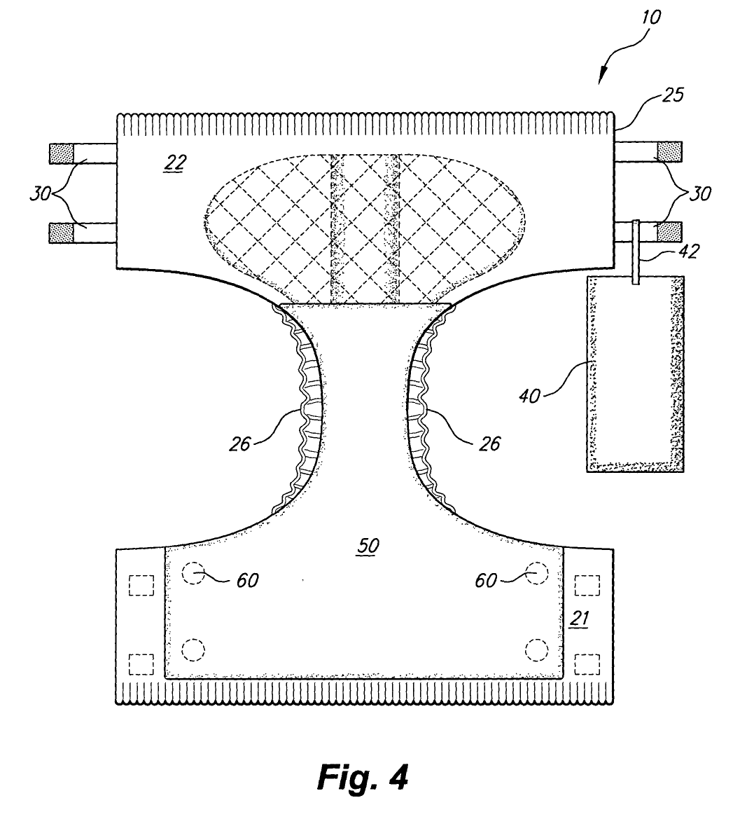

FIG. 4 illustrates an additional second generally flat and rectangular layer 50 which is placed together with the generally flat and rectangular diaper 20. The second generally flat and rectangular layer 50 is secured together with the generally flat and rectangular diaper 20 using a second plurality of hook and loop fasteners 60 to provide an additional layer of protection and strength for the user U.

Use of the disposable potty garment 10 is straightforward. The disposable potty garment 10 can be used in the general population as part of an emergency preparedness kit. Instead of collecting waste product in a bucket, the user U could urinate or defecate into the disposable potty garment 10, remove the soiled disposable potty garment 10, neatly fold it and secure it into a ball and dispose it in the provided plastic bag 40. The disposable potty garment 10 can also be used by soldiers while in the field. The disposable potty garment 10 allows a soldier to discretely defecate or urinate into the disposable potty garment 10 instead of having to abandon his or her post for going to the bathroom. The disposable potty garment 10 can also be used in hospitals to replace bedpans. The advantage that the disposable potty garment 10 has over using bedpans is that it is less cumbersome and the user U can utilize it without the aid of a healthcare worker. The disposable potty garment 10 is versatile in that it can be used as an undergarment or it can be slipped into place only when intended for use.

The inside of the generally flat and rectangular diaper 20 of the disposable potty garment 10 is made of absorbent cotton material, while the outside of the generally flat and rectangular diaper 20 is made of material that does not leak, but is breathable. Such materials are not novel and are well-known to those that are schooled in the related art. The elastic gathers 26 are also tapered for added comfort and better fit. The first plurality of hook and loop fasteners 30 are applied to the disposable potty garment 10 manually once the disposable potty garment 10 is positioned around the buttocks and groin areas of the user U, as previously described.

It is to be understood that the present invention is not limited to the embodiments described above, but encompasses any and all embodiments within the scope of the following claims.

1. A disposable potty garment, worn by a user with an upper leg area, a crotch area, a buttocks area, and two side hip areas, to defecate and urinate into, comprising:
a generally flat and rectangular diaper that has a front end, a back end, a left side edge with a center area and a right side edge with a center area;
said diaper is fitted with elastic gathers along the left side edge of the center area and the right side edge of the center area to accommodate and comfortably fit around the user's upper leg area; and
the front end of the generally flat and rectangular diaper being pulled up to the user's groin area while the rear end of the generally flat and rectangular diaper is wrapped back around underneath the user's buttocks area, said front end of the generally flat and rectangular diaper held together with the rear end of the generally flat and rectangular diaper on the side hip areas of the user with a first plurality of hook and loop fasteners securing the disposable potty garment to the user.

2. The disposable potty garment according to claim 1, wherein the generally flat and rectangular diaper can be folded up into a ball and held tightly together with the first plurality of hook and loop fasteners.

3. The disposable potty garment according to claim 2, wherein the generally flat and rectangular diaper holds any human waste defecated and urinated into by the user.

4. The disposable potty garment according to claim 3, wherein the folded up generally flat and rectangular diaper and the defecated and urinated human waste is disposed of in a plastic bag.

5. The disposable potty garment according to claim 1, wherein a second generally flat and rectangular layer is placed together with the generally flat and rectangular diaper.

6. The disposable potty garment according to claim 5, wherein the second generally flat and rectangular layer is secured together with the generally flat and rectangular diaper using a second plurality of hook and loop fasteners.
